(19) United States
(12) Patent Application Publication
(10) Pub. No.: US 2010/0159600 A1
(43) Pub. Date: Jun. 24, 2010

(54) METHOD OF CONTROLLING FLUID FLOW IN MICROFLUIDIC DEVICE AND MICROFLUIDIC ANALYSIS APPARATUS

(75) Inventors: Dong-Ho SHIN, Daejeon (KR); Minsuk JEONG, Jeongeup-si (KR); Kyu Ho SONG, Seoul (KR); HyeYoon KIM, Daejeon (KR); JuHyun JEON, Daejeon (KR); Moon Youn JUNG, Daejeon (KR); Seon Hee PARK, Daejeon (KR)

Correspondence Address: AMPACC Law Group, 3500 188th Street S.W., Suite 103, Lynnwood, WA, 98037, US

(73) Assignee: Electronics and Telecommunications Research Institute, Daejeon (KR)

(21) Appl. No.: 12/581,106

(22) Filed: Oct. 16, 2009

(30) Foreign Application Priority Data
Dec. 23, 2008 (KR) 10-2008-0131856

(51) Int. Cl. G01N 35/10; G01N 33/00

(52) U.S. Cl. 436/43; 422/68.1

(57) ABSTRACT

Provided are methods of controlling a fluid flow in a microfluidic device and a microfluidic analysis apparatus. According to the method, an inclination of the microfluidic device with respect to a horizontal direction can be adjusted to simply and accurately control a fluid flow in the microfluidic device. Thus, a fluid conveyance can be completely controlled, and an inspection can be performed using only a small amount of sample. In addition, the method of controlling the fluid flow in the microfluidic device can be manually performed by a user. Thus, since a power is not required, the method of controlling the fluid flow in the microfluidic device can be economical and simple. The microfluidic analysis apparatus includes an inclination operation unit for causing an inclination change of a receiving part of the microfluidic device with respect to a horizontal plane and an inclination control part for controlling an operation of the inclination operation unit to simply and accurately control the fluid flow, thereby to accurately analyze the fluid.

### CROSS-REFERENCE TO RELATED APPLICATIONS

This U.S. non-provisional patent application claims priority under 35 U.S.C. §119 of Korean Patent Application No. 10-2008-0131856, filed on Dec. 23, 2008, the entire contents of which are hereby incorporated by reference.

### BACKGROUND OF THE INVENTION

The present invention disclosed herein relates to a method of controlling a fluid flow in a microfluidic device and a microfluidic analysis apparatus.

Typical biochips quickly detect analytes such as biomaterials or environmental materials using membranes or hygroscopic papers. For example, biochips may be used for pregnancy diagnostic apparatuses, apparatuses for measuring hormones such as human chorionic gonadotropin (HCG), and alpha fetoprotein (AFP) (this is used as a liver cancer biomarker) measurements, which are currently commercialized. Biochips realized using membranes are disadvantageous for adjusting the flow of microfluid. The membranes may be formed of a polymeric material having a predetermined thickness and pores. Thus, the membranes are used for a simple test that determines whether an analyte is present at a previously set concentration or more. However, uniformity of sizes of the pores formed in the membranes is below the level required for an accurate test. In addition, if the sizes of the pores within the membrane are determined, strength of a capillary force is also determined. As a result, since it is impossible to adjust a microfluidic flow velocity in the membrane, the biochip realized using the membrane is not suitable for testing to accurately quantify the concentration of an analyte.

Alternatively, to overcome such limitations, there is a method which uses a microchannel as a passage for transferring a fluid. Based on hydrodynamics, a technology is widely used, which adjusts microfluidic flow velocity using microchannels with different widths and depths, and configurations of the microchannels are controlled to increase and decrease capillary force. There are many limitations in a typical biochip field in which a constant transfer velocity of the fluid in a microchannel, a constant reaction time in a reaction region, and a transfer stopping ability of fluid are necessary for quantifying analytes. Biochips using only capillary action are limited in that only the configuration and size of a channel are adjusted to accurately control fluid flow. Although a method in which an inner wall of the channel is surface-treated to have a hydrophilic property or a hydrophobic property to control the fluid was attempted, it is difficult to realize a biochip having a function that stops the fluid at a desired position and transfers the fluid to a desired position. For example, there is a technique in which a hydrophilic region of a capillary is defined to prevent the fluid from flowing so as to maintain constant reaction time. When the fluid reaches the hydrophilic region, the fluid flow is stopped due to a property in which the inner wall of the channel pushes the fluid. In general, most materials tend to have the hydrophilic property at the initial stage. However, if the materials contact the fluid for a long time, the materials tend to convert to the hydrophobic property. Thus, the fluid passes through the hydrophilic region at a very slow speed. During this time, the fluid is stopped in the channel in proportion to the surface area of the hydrophilic region. When the reaction time is controlled using such a method, a specific section of the channel should have the hydrophobic property. Thus, a suitable material and a processing method should be contrived in consideration of a physicochemical property of the fluid to be used. Also, in the case where the hydrophilic region of the channel is defined to prevent the fluid from flowing so as to maintain constant reaction time, there is a limitation that the hydrophilic property is insecure in the hydrophilic region due to absorption of atmospheric moisture, the amount of a reaction material, and inertial force of fluid flow in the reaction region. As a result, the reaction material within the reaction region may flow into the hydrophilic region.

There is a method in which pressure and electric energy are used to transfer and treat a fluid within a microfluidic device. In the case where pressure is used, a separate pressure regulator (e.g., a syringe pump or a peristaltic pump) is required, and the volume of a diagnosis system including a microfluidic device increases. In addition, the system price is affected by the pressure regulator more than the microfluidic device. Thus, this technique is unsuited for a point of care system (POCS) market in which a microfluidic device having a small size and a low price is required. On the other hand, it is advantageous that a relatively small system may be used in a method in which a fluid flow is controlled using an electrokinetic technique when compared to the method using pressure. However, a method for controlling the fluid using the electric energy may be used very limitedly. To apply the electric energy, an electrode should be provided in the microfluidic device. Accordingly, a unique configuration and method should be provided according to a property of the fluid, and various devices for transferring an electrical signal into the microfluidic device should be disposed in combination. Thus, it is complicated to manufacture and realize the system even if the system is small. Specifically, when many reaction processes are performed in one microfluidic device, the electric energy should be adjusted for each process in the case where the electrical property of the fluid is changed in each process. Therefore, the above-described method may be very complicated and difficult.

### SUMMARY OF THE INVENTION

The present invention provides a method of controlling a fluid flow in a microfluidic device that can simply and accurately control the fluid flow.

The present invention also provides a microfluidic analysis apparatus that can simply and accurately control a fluid flow to accurately analyze a fluid.

Embodiments of the present invention provide methods of controlling a fluid flow in a microfluidic device having an upper plate, a lower plate facing the upper plate, and a flow path defined between the upper plate and the lower plate, the methods including: adjusting an inclination of the microfluidic device with respect to a horizontal direction to control the fluid flow.

In some embodiments, the methods may further include inclining the microfluidic device so that an end of the microfluidic device is disposed downward from a direction in which the fluid flows along the flow path to accelerate the fluid flow.

In other embodiments, the methods may further include inclining the microfluidic device so that an end of the microfluidic device is disposed upward from a direction in which the fluid flows along the flow path to decelerate or stop the fluid flow.

In still other embodiments, the lower plate may further include a detection part disposed in a predetermined region of the flow path to detect a specific material contained in the fluid, and the methods may further include: inclining the microfluidic device so that an end of the microfluidic device is disposed downward from the fluid flow direction to accelerate the fluid flow until the fluid reaches the detection part to fully fill the detection part; and when the detection part is fully filled with the fluid, inclining the microfluidic device so that an end of the microfluidic device is disposed upward from a direction in which the fluid flows along the flow path to decelerate or stop the fluid flow.

In even other embodiments, the inclining of the microfluidic device so that an end of the microfluidic device is disposed upward from a direction in which the fluid flows along the flow path to decelerate or stop the fluid flow when the detection part is fully filled with the fluid may proceed until an reaction for detecting the specific material is completed in the detection part.

In yet other embodiments, after the reaction for detecting the specific material is completed in the detection part, the methods may further include inclining the microfluidic device so that an end of the microfluidic device is disposed downward from the fluid flow direction to accelerate the fluid flow.

In further embodiments, the lower plate may further include a reaction part coated with a reaction material reacting with a predetermined material contained in the fluid to generate a specific material to be detected by the detection part, and the method may further include: inclining the microfluidic device so that an end of the microfluidic device is disposed downward from the fluid flow direction to accelerate the fluid flow until the fluid reaches the reaction part to fully fill the reaction part; and when the reaction part is fully filled with the fluid, inclining the microfluidic device so that an end of the microfluidic device is disposed upward from a direction in which the fluid flows along the flow path to decelerate or stop the fluid flow.

In still further embodiments, the inclining of the microfluidic device so that an end of the microfluidic device is disposed upward from a direction in which the fluid flows along the flow path to decelerate or stop the fluid flow when the detection part is fully filled with the fluid may proceed until an reaction of the predetermined material and the reaction material is completed in the reaction part.

In other embodiments of the present invention, microfluidic analysis apparatuses include: a microfluidic device including an upper plate, a lower plate facing the upper plate, a flow path defined between the upper plate and the lower plate, and a detection part disposed in a predetermined region of the flow path on a surface of the lower plate to detect a specific material contained in a fluid: a microfluidic device receiving part receiving the microfluidic device; an inclination operation unit causing an inclination change of the microfluidic device receiving part with respect to a horizontal plane; an inclination control part controlling an operation of the inclination operation unit; and a reading part reading a data value with respect to a specific material in the detection part.

In some embodiments, the inclination operation unit may include an elevating actuator respectively supporting at least both ends of the microfluidic device or a hinge disposed at a central portion of the microfluidic device receiving part.

In other embodiments, the microfluidic analysis apparatuses a fixing unit fixing the microfluidic device. At this time, the fixing unit may include a robot arm, a belt, or an adhesive.

In still other embodiments, the microfluidic device may further include a lower plate electrode electrically connected to the detection part and disposed in a predetermined region of the lower plate, and the microfluidic analysis apparatuses may further include a receiving part electrode disposed in a predetermined region of the microfluidic device receiving part, contacting with the lower plate electrode, and electrically connected to the reading part.

### BRIEF DESCRIPTION OF THE FIGURES

The accompanying figures are included to provide a further understanding of the present invention, and are incorporated in and constitute a part of this specification. The drawings illustrate exemplary embodiments of the present invention and, together with the description, serve to explain principles of the present invention. In the figures:

FIGS. 1A, 1B, and 1C are cross-sectional views illustrating force vectors acting on a microfluid according to inclination of a microfluidic device;

FIGS. 2 through 7 are cross-sectional views illustrating sequentially a method of controlling a fluid flow in a microfluidic device according to an embodiment of the present invention;

FIG. 8 is a schematic cross-sectional view of a microfluidic analysis apparatus according to an embodiment of the present invention; and

FIG. 9 is a schematic cross-sectional view of a microfluidic analysis apparatus according to another embodiment of the present invention.

### DETAILED DESCRIPTION OF PREFERRED EMBODIMENTS

Preferred embodiments of the present invention will be described below in more detail with reference to the accompanying drawings. The present invention may, however, be embodied in different forms and should not be construed as limited to the embodiments set forth herein. Rather, these embodiments are provided so that this disclosure will be thorough and complete, and will fully convey the scope of the present invention to those skilled in the art.

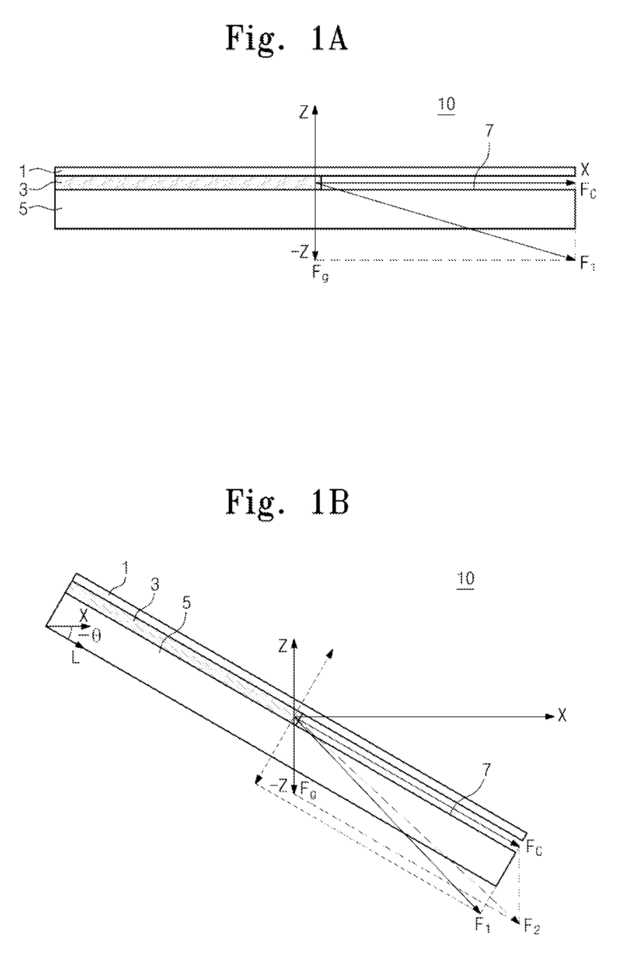

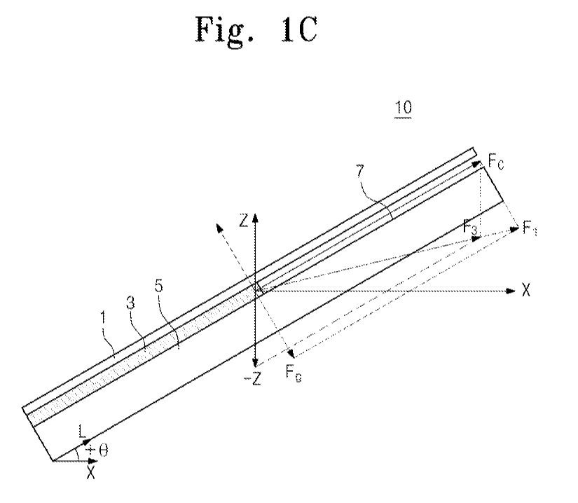

FIGS. 1A, 1B, and 1C are cross-sectional views illustrating force vectors acting on a microfluid according to inclination of a microfluidic device.

Referring to FIG. 1A, a microfluidic device 10 includes an upper plate 1, a lower plate 5 facing the upper plate 1, and a fluid 3 flowing along a flow path 7 defined by the upper plate 1 and the lower plate 5. The microfluidic device 10 is disposed in parallel to a horizontal direction (X-axis). A force F₁ applied to the fluid 3 may be expressed as a resultant force of a capillary force F_{c} acting in parallel to the horizontal direction (X-axis) and a gravity F_{g} acting in a direction perpendicular to the horizontal direction.

FIG. 1B illustrates force vectors acting on the microfluid when the microfluidic device 10 of FIG. 1A is inclined at an angle −θ. The inclination of the microfluidic device 10 may be defined as an angle between the horizontal direction (X-axis direction) and a fluid flow direction within the flow path 7. If the flow path 7 is parallel to a bottom surface of the lower plate 5, the inclination of the microfluidic device 10 may be defined as an angle θ between the horizontal direction (X-axis direction) and a length direction (L-axis direction) of the lower plate 5. The microfluidic device 10 inclined at the “angle −θ” denotes that the fluid flow direction is inclined downward at the angle B from the horizontal direction. At this time, as shown in FIG. 1B, a force F₂ applied to the fluid 3 within the microfluidic device 10 may be expressed as a resultant force of a capillary force F_{c} inclined downward from the horizontal direction (X-axis) and the gravity F_{g} acting in the direction perpendicular to the horizontal direction. It may be seen that the resultant force F₂ of FIG. 1B is greater than the resultant force F₁ of FIG. 1A.

$$F = m \frac{d V}{d t} \qquad ( 1 )$$

where the force F is equal to the product of the mass m and the change in velocity V over time t, as shown in Equation 1. Thus, if the microfluidic device 10 is inclined downward from the fluid flow direction, the resultant force acting on the fluid 3 increases from F₁ to F₂. As a result, the fluid flow velocity increases. Accordingly, when the microfluidic device 10 is inclined downward from the fluid flow direction, the fluid flow velocity may increase.

A contrary case will be described below with reference to FIG. 1C.

FIG. 1C illustrates force vectors acting on the microfluid when the microfluidic device 10 of FIG. 1A is inclined at an angle +θ. The microfluidic device 10 inclined at the “angle +θ” denotes that the fluid flow direction is inclined upward at the angle θ from the horizontal direction. At this time, as shown in FIG. 1C, a force F₃ applied to the fluid 3 within the microfluidic device 10 may be expressed as a resultant force of a capillary force F_{c} inclined upward from the horizontal direction (X-axis) and the gravity F_{g} acting in the direction perpendicular to the horizontal direction. It may be seen that the resultant force F₃ of FIG. 1C is less than the resultant force F₁ of FIG. Thus, referring to Equation 1, if the microfluidic device 10 is inclined upward from the fluid flow direction, the resultant force acting on the fluid 3 decreases from F₁ to F₃. As a result, the fluid flow velocity decreases. Accordingly, when the microfluidic device 10 is inclined upward from the fluid flow direction, the fluid flow velocity may decrease.

If the microfluidic device 10 is inclined at +90°, the resultant force F₄ applied to the fluid 3 is a difference value between the capillary force Fc and the gravity F_{g}. If the capillary force F_{c} is equal to the gravity Fₛ, a force applied to the fluid 3 is zero to stop the fluid flow.

Therefore, the inclination of the microfluidic device 10 may be adjusted with respect to the horizontal direction to control the flow velocity of the fluid 3 within the microfluidic device 10.

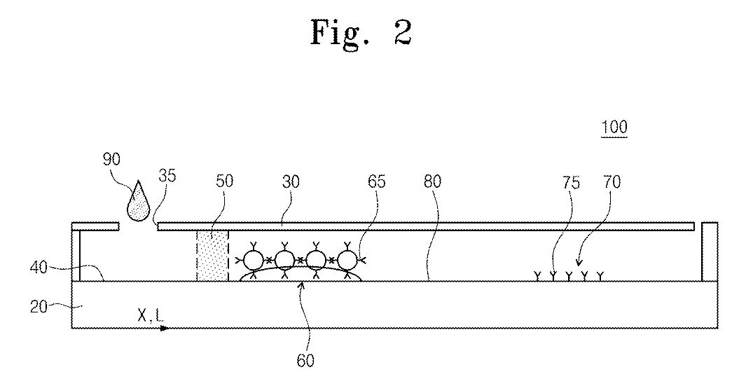

A method of controlling a flow velocity of a fluid within a microfluidic device according to an embodiment will be described in detail with reference to FIGS. 2 through 7.

Referring to FIG. 2, a microfluidic device 100 according to this embodiment includes an upper plate 30 and a lower plate 20 facing the upper plate 30. A fluid injection hole 35 through which a fluid 90 is injected is defined in a predetermined region of the upper plate 30. A storage chamber 40 is disposed in a region, which overlaps with the fluid injection hole 35, between the upper plate 30 and the lower plate 20. In this embodiment, the fluid 90 may include blood. A filter 50 for filtering macromolecules such as a white blood corpuscle or a red blood corpuscle within the blood is disposed at a lateral side. A flow path 80 is defined between the upper plate 30 and the lower plate 20. A reaction part 60 is disposed in a predetermined region of the flow path 80 adjacent to the filter 50. For example, a water soluble paste or a hydrophilic polymer may be coated on the reaction part 60. Polystyrene nanoparticles to which a detection antibody 65 for detecting a predetermined material such as an antigen within the blood is fixed may be attached to the reaction part 60 coated with the water soluble paste or the hydrophilic polymer. Fluorescent nanoparticles or gold nanoparticles may be attached to the detection antibody 65.

A detection part 70 to which a capture antibody 75 for capturing the detection antibody 65 is fixed may be disposed in a predetermined region of the flow path 80 spaced apart from the reaction part 60. In the microfluidic device 100, the fluid 90 is injected into the storage chamber 40 through the fluid injection hole 35. In this embodiment, a length direction of the flow path 80 is parallel to a length direction L of a bottom surface of the lower plate 20. At this time, the microfluidic device 100 may be in a horizontal state. That is, a horizontal direction (X-axis) is equal to the length direction L of the bottom surface of the lower plate 20.

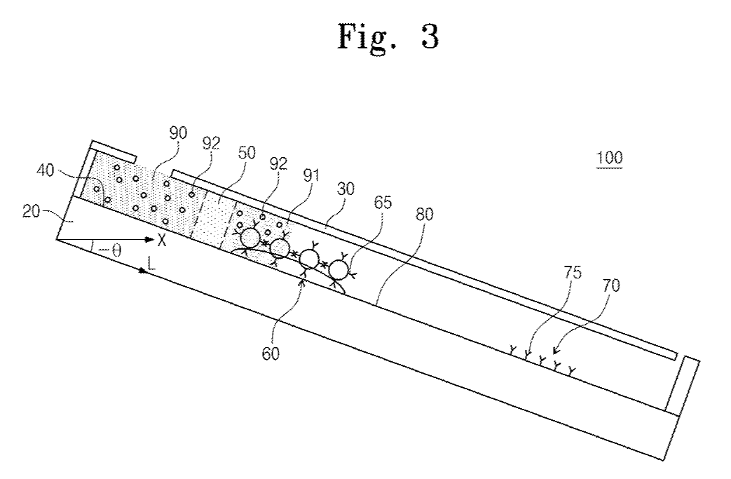

Referring to FIG. 3, antigens 92 with respect to a specific disease may exist in the blood 90 that is the fluid 90 stored in the storage chamber 40. The antigens 92 having a small particle pass through the filter 50 together with blood plasma 91 to reach the reaction part 60. The microfluidic device 100 is inclined downward from a flow direction of the blood plasma 91 until the reaction part 60 is fully filled with the blood plasma 91 and the antigens 92. That is, the length direction L of the lower plate 20 is inclined at an angle −θ from the horizontal direction (X-axis direction). Thus, the blood plasma 91 may quickly reach the reaction part 60.

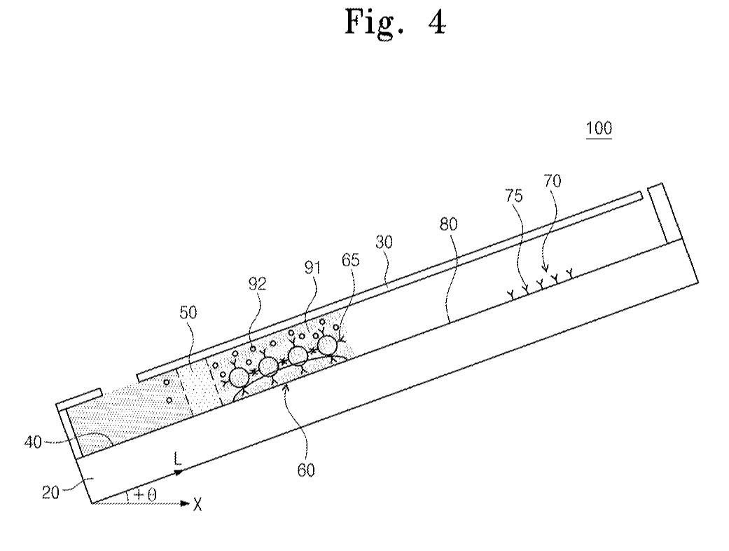

Referring to FIG. 4, when the reaction part 60 is fully filled with the blood plasma 91 and the antigens 92, the microfluidic device 100 is inclined upward from a flow direction of the blood plasma 91. That is, the length direction L of the lower plate 20 is inclined at an angle +θ from the horizontal direction (X-axis direction). As a result, a flow velocity of the blood plasma 91 may be reduced, or a flow of the blood plasma 91 may be stopped. Thus, the blood plasma 91 stays in the reaction part 60 for a long time to sufficiently cause an antigen-antibody reaction of the antigens 92 and the detection antibody 65 in the reaction part 60.

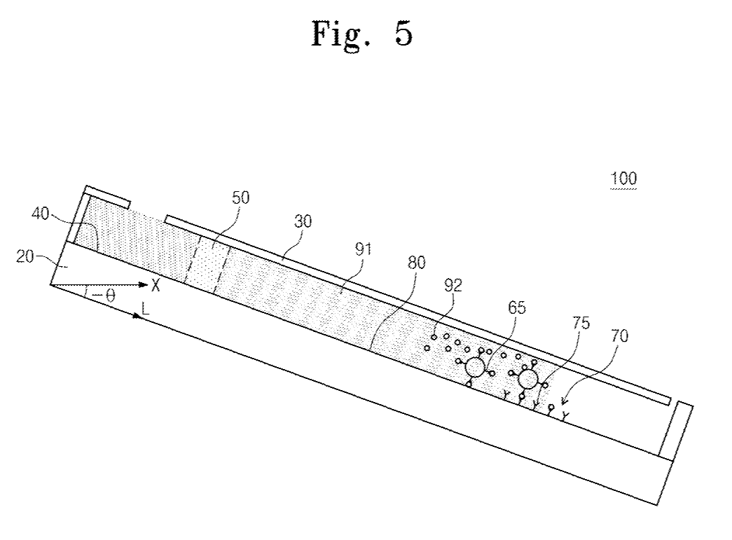

Referring to FIG. 5, when the antigen-antibody reaction of the antigens 92 and the detection antibody 65 is sufficiently performed in the reaction part 60 to sufficiently produce an antigen-detection antibody complex, the microfluidic device 100 is inclined downward from the flow direction of the blood plasma 91. During the antigen-antibody reaction within the reaction part 60, the water soluble paste or the hydrophilic polymer coated on the reaction part 60 is fully dissolved in water. Thus, the water soluble paste or the hydrophilic polymer is not illustrated in FIGS. 5 through 9. That is, the length direction L of the lower plate 20 is inclined at the angle −θ from the horizontal direction (X-axis). Thus, the blood plasma 91 and the antigens 92 may quickly reach the reaction part 60.

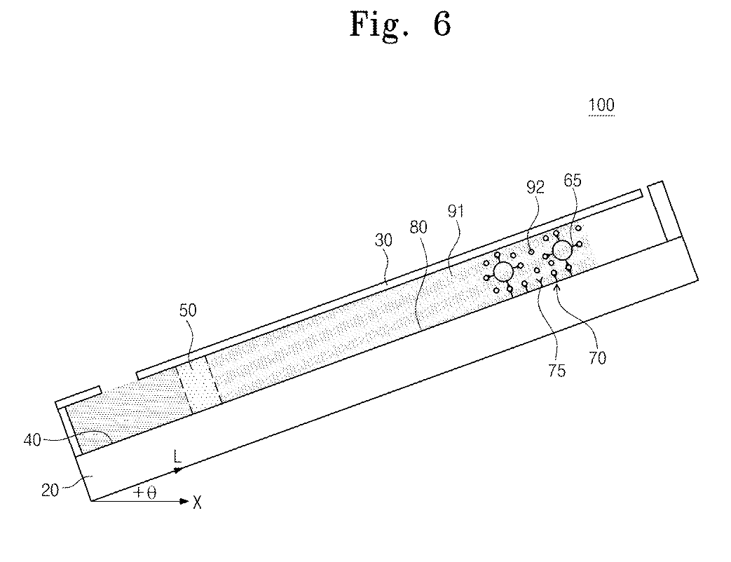

Referring to FIG. 6, when the detection part 70 is filly filled with the blood plasma 91, the microfluidic device 100 is inclined upward from the flow direction of the blood plasma 91. That is, the length direction L of the lower plate 20 is inclined at the angle +θ from the horizontal direction (X-axis). As a result, a flow velocity of the blood plasma 91 may be reduced, or a flow of the blood plasma 91 may be stopped. Thus, the blood plasma 91 stays in the detection part 70 for a long time to sufficiently cause an antigen-antibody reaction of the antigen-detection antibody complex and the capture antibody 75 in the detection part 70. As a result, the antigen-detection antibody complex is fixed to the capture antibody 75.

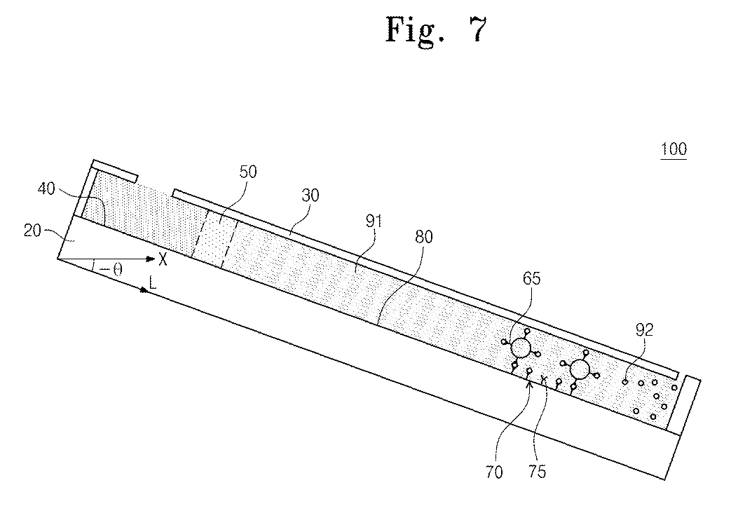

Referring to FIG. 7, when the antigen-antibody reaction of the antigen-detection antibody complex and the capture antibody 75 is sufficiently performed in the detection part 70 and the antigen-detection antibody complex is sufficiently fixed to the capture antibody 75, the microfluidic device 100 is inclined downward from the flow direction of the blood plasma 91. That is, the length direction L of the lower plate 20 is inclined at the angle −θ from the horizontal direction (X-axis). Thus, materials that are not fixed to the capture antibody 75 may be quickly removed. As a result, a washing process may be omitted.

According to the above-described processes, fluid conveyance may be completely controlled, and the antigen-antibody immunity reaction may be realized using only a small amount of sample. In addition, the processes may be manually performed by a user. That is, the user may identify a position of the fluid with the naked eye and adjust the inclination of the microfluidic device 100 using a user's finger to control the fluid flow velocity. In this case, the upper plate 30 may be formed of a transparent material.

In the inclination of the microfluidic device 100 described with reference to FIGS. 3 through 7, the angle θ may be changed as necessary. For example, the microfluidic device 100 may be inclined at about −30° in FIG. 3, about +50° in FIG. 4, about −40° in FIG. 5, +70° in FIG. 6, and about −50° in FIG. 7.

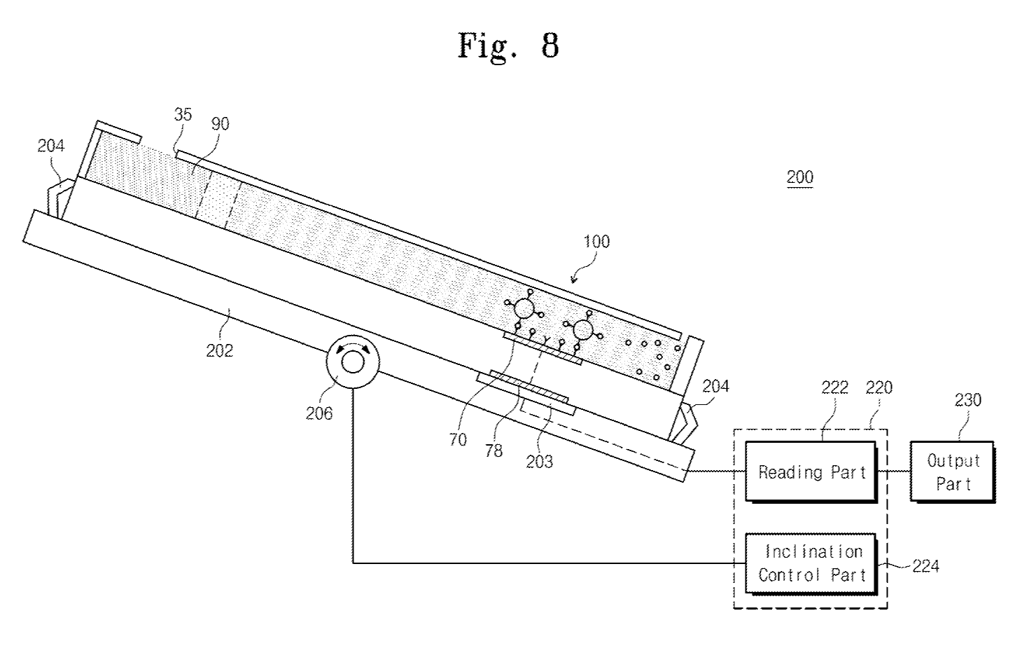

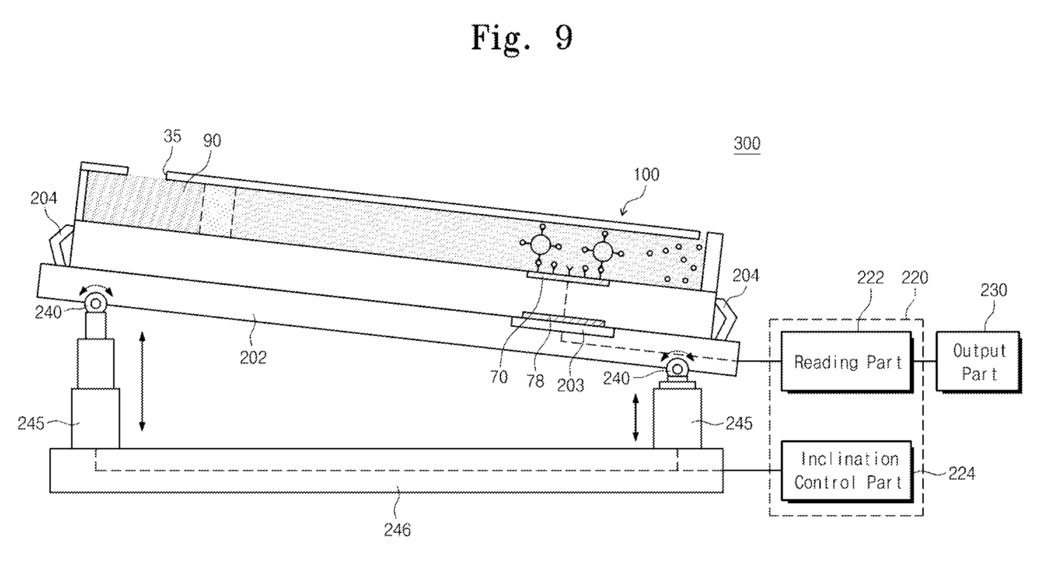

A microfluidic analysis device in which a fluid flow velocity can be automatically adjusted, but manually adjusted, will be described with reference to FIGS. 8 and 9. FIG. 8 is a schematic cross-sectional view of a microfluidic analysis apparatus according to an embodiment of the present invention. FIG. 9 is a schematic cross-sectional view of a microfluidic analysis apparatus according to another embodiment of the present invention. The microfluidic analysis apparatuses of FIGS. 8 and 9 detect and read a specific material contained in a fluid using an electrochemical method.

Referring to FIGS. 8 and 9, a loaded microfluidic device 100 includes a lower plate electrode 78 electrically connected to a detection part 70. A detection antibody 65 to which a gold nanoparticle is fixed may be coated on a reaction part 60 of the microfluidic device 100, and a detection antibody-antigen complex to which the gold nanoparticle is fixed may be fixed to the detection part 70. According to the electrochemical method, the specific material may be detected and read using a property in which conductivity increases due to the gold nanoparticle as the number of complex increases.

Referring to FIG. 8, the microfluidic analysis equipment 200 according to this embodiment may include a receiving part 202 to which the microfluidic device 100 is loaded. A fixing unit 204 for fixing the microfluidic device 100 is disposed in the receiving part 202. A receiving part electrode 203 contacting with the lower plate electrode 78 of the microfluidic device 100 may be disposed in a predetermined region of the receiving part 292. The fixing unit 204 illustrated in FIG. 8 may include a robot arm, but it is not limited thereto. For example, the fixing unit 204 may include various devices such as a belt or an adhesive. A hinge part 206 for providing a rotation shaft is disposed at a central portion of the receiving part 202. The hinge part 206 is connected to a drive motor (not shown). The microfluidic analysis apparatus 200 may further include a central processing unit 220 (CPU). The CPU 220 may include an inclination control part 224 for controlling a rotation angle of the hinge part 206 and a reading part 222 electrically connected to the receiving part electrode 203 to read a data value (e.g., such as conductivity) of a specific material detected by the detection part 70. The data value read by the reading part 222 may be outputted by an output part 230.

A process for utilizing the microfluidic analysis apparatus 200 will be described below.

The microfluidic device 100 in which a fluid 90 illustrated in FIG. 2 is dropped through a fluid injection hole 35 is loaded on the receiving part 202 of the microfluidic analysis apparatus 200. Then, the fixing unit 204 such as the robot arm fixes the microfluidic device 100. An inclined angle, a process sequence, and a time for maintaining the inclined angle may be previously programmed in the inclination control part 224 according to the sequence described with reference to FIGS. 3 through 7. Thus, the hinge part 206 is rotated at a predetermined angle in left and right directions according to a signal of the inclined control part 224. As a result, an antigen-antibody reaction may sufficiently occur in a reaction part 60 and the detection part 70 within the microfluidic device 100. FIG. 8 illustrates the microfluidic analysis apparatus 200 including the microfluidic device 100 illustrated in FIG. 7 which the inclination of the microfluidic device 100 is completely adjusted according to the sequence. Sequentially, an antigen-detection antibody complex is sufficiently fixed to a capture antibody in the detection part 70, and then, the data value such as the conductivity is read by the reading part 222 to output the read data value through the output part 230.

The microfluidic analysis apparatus 300, to which the same microfluidic device 100 as that of FIG. 8 is loaded, different from the microfluidic analysis apparatus 200 of FIG. 8 will be described with reference to FIG. 9.

Referring to FIG. 9, the microfluidic analysis apparatus 300 according to this embodiment includes an elevating actuator 245 respectively connected to both ends of a receiving part 202 and vertically elevated. The elevating actuator 245 may be connected to the receiving part 204 using a hinge 240. At least two elevating actuators 245 may be fixed to a support 246. The microfluidic analysis apparatus 300 according to this embodiment does not include the hinge part 206, illustrated in FIG. 8, for providing a rotation shaft at a central portion of the receiving part 202. According to the microfluidic analysis apparatus 300, an inclination of the receiving part 202 is determined according to levels of the elevating actuators 245. The microfluidic analysis apparatus 300 further includes a CPU 220. An inclination control part 224 included in the CPU 220 controls the levels of the elevating actuators 245. Other components and an operation method of the microfluidic analysis apparatus 300 according to this embodiment are equal to those of the microfluidic analysis apparatus 200 of FIG. 8.

Although the microfluidic analysis apparatuses 200 and 300 detect and read the specific material using the electrochemical method, the present invention is not limited thereto. For example, the microfluidic analysis apparatuses 200 and 300 may detect and read the specific material using a laser-induced fluorescence detection. That is, the microfluidic analysis apparatus adopting the laser-induced fluorescence detection, e.g., a fluorescent scanner may include a fixing unit for fixing the microfluidic device according to the present invention, an inclination operation unit for changing an inclination of a receiving part with respect to a horizontal direction, and an inclination control part for controlling an operation state of the inclination operation unit.

In the method of controlling the fluid flow in the microfluidic device according to an embodiment of the present invention, the inclination of the microfluidic device with respect to the horizontal direction can be adjusted to control a correlation between the capillary force and the effective gravity, which act on the microfluid within the flow path. Thus, the fluid flow can be simply and accurately controlled in the microfluidic device. In addition, the fluid conveyance may be completely controlled, and the inspection can be performed using only a small amount of sample. The method of controlling the fluid flow in the microfluidic device can be manually performed by the user. That is, the user can identify a position of the fluid with the naked eye and adjust the inclination of the microfluidic device 100 using the user's finger to control the fluid flow velocity. Thus, since a power is not required, the method of controlling the fluid flow in the microfluidic device can be economical and simple.

The microfluidic analysis apparatus according to another embodiment of the present invention can include the inclination operation unit for causing the inclination change of the receiving part of the microfluidic device with respect to the horizontal plane and the inclination control part for controlling the operation of the inclination operation unit to simply and accurately control the fluid flow, thereby to accurately analyze the fluid.

The above-disclosed subject matter is to be considered illustrative, and not restrictive, and the appended claims are intended to cover all such modifications, enhancements, and other embodiments, which fall within the true spirit and scope of the present invention. Thus, to the maximum extent allowed by law, the scope of the present invention is to be determined by the broadest permissible interpretation of the following claims and their equivalents, and shall not be restricted or limited by the foregoing detailed description.

What is claimed is:

1. A method of controlling a fluid flow in a microfluidic device having an upper plate, a lower plate facing the upper plate, and a flow path defined between the upper plate and the lower plate, the method comprising:
adjusting an inclination of the microfluidic device with respect to a horizontal direction to control the fluid flow.

2. The method of claim 1, further comprising accelerating the fluid flow by inclining the microfluidic device so that an end of the microfluidic device is disposed downward from a direction in which the fluid flows along the flow path.

3. The method of claim 1, further comprising decelerating or stopping the fluid flow by inclining the microfluidic device so that an end of the microfluidic device is disposed upward from a direction in which the fluid flows along the flow path.

4. The method of claim 1, wherein the lower plate further comprises a detection part disposed in a region of the flow path to detect a specific material contained in the fluid, and
the method further comprising:
accelerating the fluid flow until the fluid reaches the detection part and fully fills the detection part by inclining the microfluidic device so that an end of the microfluidic device is disposed downward from the fluid flow direction; and
decelerating or stopping the fluid flow in the detection part by inclining the microfluidic device so that an end of the microfluidic device is disposed upward from a direction in which the fluid flows along the flow path, when the detection part is fully filled with the fluid.

5. The method of claim 4, wherein the decelerating or stopping of the fluid flow in the detection part proceeds until a reaction for detecting the specific material is completed in the detection part.

6. The method of claim 5, after the reaction for detecting the specific material is completed in the detection part, further comprising accelerating the fluid flow by inclining the microfluidic device so that an end of the microfluidic device is disposed downward from the fluid flow direction.

7. The method of claim 4, wherein the lower plate further comprises a reaction part coated with a reaction material reacting with a predetermined material contained in the fluid to generate a specific material to be detected by the detection part, and
the method further comprising:
accelerating the fluid flow until the fluid reaches the reaction part to fully fill the reaction part by inclining the microfluidic device so that an end of the microfluidic device is disposed downward from the fluid flow direction; and
decelerating or stopping the fluid flow in the reaction part by inclining the microfluidic device so that an end of the microfluidic device is disposed upward from a direction in which the fluid flows along the flow path, when the reaction part is fully filled with the fluid.

8. The method of claim 7, wherein the decelerating or stopping of the fluid flow in the reaction part proceeds until an reaction of the predetermined material and the reaction material is completed in the reaction part.

9. A microfluidic analysis apparatus comprising:
a microfluidic device comprising an upper plate, a lower plate facing the upper plate, a flow path defined between the upper plate and the lower plate, and a detection part disposed in a region of the flow path on a surface of the lower plate to detect a specific material contained in a fluid;
a microfluidic device receiving part receiving the microfluidic device;
an inclination operation unit causing an inclination change of the microfluidic device receiving part with respect to a horizontal plane;
an inclination control part controlling an operation of the inclination operation unit; and
a reading part reading a data value with respect to a specific material in the detection part.

10. The microfluidic analysis apparatus of claim 9, wherein the inclination operation unit comprises elevating actuators respectively supporting at least both ends of the microfluidic device or a hinge disposed at a central portion of the microfluidic device receiving part.

11. The microfluidic analysis apparatus of claim 9, further comprising a fixing unit fixing the microfluidic device.

12. The microfluidic analysis apparatus of claim 11, wherein the fixing taut comprises a robot arm, a belt, or an adhesive.

13. The microfluidic analysis apparatus of claim 9, wherein the microfluidic device further comprises a lower plate electrode electrically connected to the detection part and disposed in a region of the lower plate, and
the microfluidic analysis apparatus further comprising a receiving part electrode disposed in a region of the microfluidic device receiving part, contacting with the lower plate electrode, and electrically connected to the reading part.
